(19) United States
(12) Patent Application Publication
(10) Pub. No.: US 2014/0105962 A1
(43) Pub. Date: Apr. 17, 2014

(54) MERRIMIA TRIDENTATA COMPOSITIONS

(71) Applicant: Vimal Patel, Winter Garden, FL (US)

(72) Inventor: Vimal Patel, Winter Garden, FL (US)

(21) Appl. No.: 14/059,125

(22) Filed: Oct. 21, 2013

Related U.S. Application Data
(63) Continuation of application No. 13/620,753, filed on Oct. 17, 2012, now abandoned.

(51) Int. Cl. A61K 36/39; A61K 36/18; A61K 36/45; A61K 36/324; A61K 36/9066; A61K 36/61

(52) U.S. Cl. CPC A61K 36/39; A61K 36/9066; A61K 36/61; A61K 36/45; A61K 36/324; A61K 36/18
USPC 424/450; 424/725; 424/756; 424/769

(57) ABSTRACT

A composition including a powder or extract of Merremia Tridentata mixed with a liposome carrier. The liposome carrier may be a lotion, cream or suspension. The composition may be used to treat Osteophytes of the spine, feet, hands, and knees.

This application is a Continuation to U.S. non-provisional application No. 13/620,753, filed Oct. 17, 2012 the contents of which are herein incorporated by reference.

### BACKGROUND OF THE INVENTION

The present invention relates to Merrimia Tridentata compositions and, more particularly, to Merrimia Tridentata mixed with a liposome carrier.

Osteophytes, commonly referred to as bones spurs, are bony projections that form along joint margins. Osteophytes form because of the increase in a damaged joint's surface area. This is most common from the onset of arthritis. Osteophytes usually limit joint movement and typically cause pain. Osteophytes form naturally on the back of the spine as a person ages and are a sign of degeneration in the spine. Bone spurs on the spine can impinge on nerves that leave the spine for other parts of the body. This impingement can cause pain in both upper and lower limbs and numbness or tingling sensations in the hands and feet because the nerves are supplying sensation to their dermatomes. Spurs can also appear on the feet, either along toes or the heel, as well as on the hands.

As can be seen, there is a need for a treatment and relief from Osteophytes.

### SUMMARY OF THE INVENTION

In one aspect of the present invention, a composition comprises Merremia Tridentata, the composition further comprising a topical carrier for an application to an external surface of an animal body, wherein the carrier comprises a liposome.

In another aspect of the present invention, a method of treatment comprises: providing a composition comprising Merremia Tridentata, the composition further comprising a topical carrier comprising a liposome; and applying the composition to an external surface of the animal body affected by Osteophytes

These and other features, aspects and advantages of the present invention will become better understood with reference to the following drawings, description and claims.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a composition including a powder or extract of Merremia Tridentata mixed with a liposome carrier. The liposome carrier may be a lotion, cream or suspension. The composition may be used to treat Osteophytes of the spine, feet, hands, and knees.

The present invention may include a composition. The composition may include the plant Merremia Tridentata. Oil may be extracted from the plant, creating Prasarini oil, which may be used in the composition. In alternative embodiments, the plant, such as the flowers, roots, and stem may be ground up to form a powder, which may be used in the composition. The composition of the present invention may further include a liposome carrier. The liposome carrier may be in the form of a lotion, cream, suspension, hormone replacement therapy cream, or any liposomal cream known in the art.

In certain embodiments, the cream of the present invention may include a liposome base cream, Prasarini oil or Merrimia Tridentata powder, and may further include at least one of Mahanarayana oil (ayurvedic oil) and Turmeric. In certain embodiments, the amount of liposome base cream may be about 1 lb and up to about 2.54 kg. If using an extract, the amount of Prasarini oil may be at about 400 ml. However, if the Merrimia Tridentata is in the powder form, 2 kg of powder may be mixed with the 2.54 kg of the liposome base cream. The cream may include up to about 300 ml of Mahanarayana oil and 2g of Turmeric.

The liposome base of the present invention may include the following ingredients: water; olive oil; Aloe Barbadensis Leaf juice; Lysolecithin; Sunflower oil; Safflower oil; Perilla ocymoides seed extract; macadamia nut oil; Isopropyl palmitate; Ascorbyl Palmitate; Sodium hydroxymethylglycinate; Cetearyl alcohol; Cetearyl glucoside; Cetyl Alcohol; Vitis Vinifera seed oil; Wheat germ glycerides; Xanthan Gum; Retinyl; Palmitate; Tocopheryl acetate; and phenoxyethanol.

Every 100 ml of the Prasarini oil may include the following: Prasarini 7.778 g; Sahachara 7.778 g; Sathavari 7.78 g; Bala 7.78 g; Atmagupta 7.78 g; Asvagandha 7.78 g; Ketaki 7.78 g; Tagara 0.039 g; Madana 0.039 g; Kushtha 0.039 g; Kesara 0.039 g; Mustaka 0.039 g; Tvak 0.039 g; Rasna 0.039 g; Saindhva 0.039 g; Pippalavu 0.039 g; Mamsi 0.039 g; Manjistha 0.039 g; Meda 0.039 g; Mahameda 0.039 g; Jivaka 0.039 g; Rsabha 0.039 g; Satapusha 0.039 g; Vyaghranakha 0.039 g; Sunti 0.039 g; Devahva 0.039 g; Kakoli 0.039 g; Ksirakakoli 0.039 g; Vaca 0.039 g; Bhallataka 0.039 g; Paya 10 ml; Masu 10 ml; Sukta 10 ml; and Mamsasarasa 10 ml.

A method of making the present invention may include the following. Add up to about 400 ml of Prasarini oil to about 1 lb of liposome base cream. The liposome base cream may be a heavy base, a light base, or a regular base cream. The composition may be mixed well until the oil is absorbed into the cream and there is no excess floating oil. Then add 2 grams of Turmeric and mix well. Additional ingredients may be added, including, but not limited to, about 15 ml of clove oil, about 10 ml of wintergreen oil, and about 10 ml of Boswelia oil.

Another method of making the present invention may include the following. Add up to about 100 ml to about 300 ml of Prasarini oil and about 100 ml to about 300 ml of Mahanarayana to about 1 lb of liposome base cream. For example, the combination may include 100 ml of Prasarini oil and 300 ml of Mahanarayana oil, 200 ml of Prasarini oil and 200 ml of Mahanarayana oil, and 300 ml of Prasarini oil and 100 ml of Mahanarayana oil. The liposome base cream may be a heavy base, a light base, or a regular base cream. The composition may be mixed well until the oil is absorbed into the cream and there is no excess floating oil. Then add 2 grams of Turmeric and mix well. Additional ingredients may be added, including, but not limited to, about 15 ml of clove oil, about 10 ml of wintergreen oil, and about 10 ml of Boswelia oil.

The present invention may be applied to the surface of a subject, such as skin, to treat Osteophytes. The subject may be an animal, such as a human. The composition may be applied to the spine, foot, hand, and knee. When applied to the spine, the composition of the present invention may treat disc degeneration, disc bulging, or a herniated disc. The present invention may be applied twice a day to ensure proper delivery of the Merremia Tridentata on affected parts of body. Merrimia Tridentata mixed with the liposome based lotion may be used to effectively aid in the delivery of the Merrimia Tridentata to the affected body part. The exchange of the calcium oxalate crystals from the composition may provide relief from the symptoms of bone spurs

Delivery of Prasarini oil and other ingredients as described above with use of liposomes or liposomal technology through the skin is outlined below. Skin is a complex entity consisting of a variety of cells and organelles, each of which has a particular function. The pathways by which the materials or drugs are absorbed and the role of the vehicle on the skin can be better understood if one is familiar with skin structure and function. Skin protects the body's organs from external environmental threats, including ultraviolet rays, and acts as a thermostat to maintain body temperature. Skin includes several different layers, each with specialized functions. The major layers include the epidermis, the dermis and hypodermis. The epidermis is a stratifying layer of epithelial cells that overlies the dermis which includes the connective tissue layer. An internal layer of adipose tissue, the hypodermis, further supports the epidermis and dermis. Various factors influence the absorption of substances through the skin. Liposomes are employed as carriers of material that aid the delivery of material through upper layers of skin to desired targeted areas. The lipid bilayer structures of liposomes mimic the barrier properties of biomembranes, and therefore they offer the potential of examining the behavior of membranes of a known composition. Thus, by altering the lipid composition of the bilayer or the material incorporated, it is possible to establish differences in membrane properties. Liposomes are microscopic lipid vesicles that are formed when thin phospholipid films or lipid cakes are hydrated and stacks of liquid crystalline bilayers become fluid and swell. Liposomes store water-soluble substances in their interiors like biological cells. The phospholipids forming these liposomes enhance the penetration of the encapsulated materials into the stratum corneum region of skin. They are biodegradable and non-toxic in nature. Liposomes enable water-soluble and water-insoluble materials to be used together in a formulation entrapping oils(Prasarini oil) or oil-soluble materials in the liposome wall. Liposomes are regarded as suitable carriers because they can serve as a depot system for the sustained release of entrapped Prasarini oil. The best demonstrable feature of liposomes is that the ingredients like Prasarini oil exhibit better stability, penetration and efficacy at lower use levels. There is no proof that liposomes penetrate intact into the stratum corneum. However, it is believed that they can deliver the Prasarini oil and the other ingredients active by adhering to the corneocyte through diffusion as a result of fusion of bilayers of liposomes with the proteins and lipids of the stratum corneum.

It should be understood, of course, that the foregoing relates to exemplary embodiments of the invention and that modifications may be made without departing from the spirit and scope of the invention as set forth in the following claims.

What is claimed is:

1. A composition comprising Merremia Tridentata, the composition further comprising a topical carrier for an application to a surface of an animal body, wherein the carrier comprises a liposome.

2. The composition of claim 1, wherein the Merremia Tridentata is in the form of at least one of an extract and a powder.

3. The composition of claim 2, wherein the extract is an oil extract.

4. The composition of claim 3, wherein the oil extract is Prasarini oil.

5. The composition of claim 4, wherein the Prasarini oil is at a concentration in the carrier of at least about 1.25 g/ml to up to about 6.35 g/ml.

6. The composition of claim 4, wherein the Prasarini oil is further mixed with Mahanarayana oil.

7. The composition of claim 6, wherein the Parasarini oil and the Maharayana oil are at a ratio of at least about 3:1 and up to about 1:3.

8. The composition of claim 4, wherein 100 ml of the Prasarini oil comprises: Prasarini 7.778 g; Sahachara 7.778 g; Sathavari 7.78 g; Bala 7.78 g; Atmagupta 7.78 g; Asvagandha 7.78 g; Ketaki 7.78 g; Tagara 0.039g; Madana 0.039 g; Kushtha 0.039 g; Kesara 0.039 g; Mustaka 0.039 g; Tvak 0.039 g; Rasna 0.039 g; Saindhva 0.039 g; Pippalavu 0.039 g; Mamsi 0.039 g; Manjistha 0.039 g; Meda 0.039 g; Mahameda 0.039 g; Jivaka 0.039 g; Rsabha 0.039 g; Satapusha 0.039 g; Vyaghranakha 0.039 g; Sunti 0.039 g; Devahva 0.039 g; Kakoli 0.039 g; Ksirakakoli 0.039 g; Vaca 0.039 g; Bhallataka 0.039 g; Paya 10 ml; Masu 10 ml; Sukta 10 ml; and Mamsasarasa 10 ml.

9. The composition of claim 1, further comprising Turmeric.

10. The composition of claim 1, further comprising clove oil, wintergreen oil, and Boswelia oil.

11. The composition of claim 1, wherein the topical carrier is in the form of a cream, lotion, or suspension.

12. The composition of claim 1, wherein the animal body is a human body.

13. The composition of claim 12, wherein the surface of the human body is an external surface of the human body.

14. A method of treatment comprising:
providing a composition comprising Merremia Tridentata, the composition further comprising a topical carrier comprising a liposome; and
applying the composition to a surface of an animal body affected by Osteophytes.

15. The method of claim 14, wherein the surface of the animal body is an external surface of the animal body.

16. The method of claim 15, wherein the animal body is a human body.

17. The method of claim 16, wherein the external surface of the human body is skin.

18. The method of claim 16, wherein the external surface of the human body affected by Osteophytes comprises at least one of the spine, foot, hand, and knee.

19. The method of claim 16, further comprising the step of applying the composition to the external surface of the human body affected by Osteophyte twice a day.
